(19) United States
(12) Patent Application Publication
(10) Pub. No.: US 2016/0348139 A1
(43) Pub. Date: Dec. 1, 2016

(54) USE OF BILE DETERGENTS TO DENATURE GLYCOPROTEINS PRIOR TO ENZYMATIC DIGESTION

(71) Applicants: Michael J. KIMZEY, Oakland, CA (US); Francis T. HAXO, San Francisco, CA (US)

(72) Inventors: Michael J. KIMZEY, Oakland, CA (US); Francis T. HAXO, San Francisco, CA (US)

(21) Appl. No.: 15/167,903

(22) Filed: May 27, 2016

Related U.S. Application Data
(60) Provisional application No. 62/168,644, filed on May 29, 2015.

(51) Int. Cl. C12P 19/04

(52) U.S. Cl. CPC C12P 19/04

(57) ABSTRACT

Bile detergents can be used to denature glycoproteins prior to enzymatically deglycosylating the glycoproteins. Bile detergents, and especially bile salts, render glycans on a glycoprotein accessible to deglycosylation enzymes, especially the enzyme PNGase F, and are compatible with the enzyme. The bile detergents can be conveniently removed by solid phase or liquid-liquid extraction techniques, and many bile detergents can be removed by acid precipitation, allowing the bile detergent to be quickly separated from the glycans, the deglycosylated protein, or both.

### CROSS-REFERENCE TO RELATED APPLICATIONS

This application claims the benefit of U.S. Provisional Application No. 62/168,644, filed May 29, 2015, the contents of which are incorporated herein by reference in their entirety.

### STATEMENT OF FEDERAL FUNDING

Not applicable.

### BACKGROUND OF THE INVENTION

This invention relates to the field of analysis of glycosylation of glycoproteins.

Many of the proteins produced by eukaryotic cells are modified after translation by the addition of covalently-linked, linear or branched chains of carbohydrates. These protein-carbohydrate conjugates are referred to as glycoproteins; the point at which the carbohydrate is attached is referred to as a glycosylation site. Attached polysaccharides or oligosaccharides are referred to as glycans. A wide range of glycans are found on the different glycosylation sites of particular glycoproteins. The particular pattern of glycans on a particular glycoprotein is determined by the specific cell line that produced the protein and the conditions under which the cells were grown.

Since the glycans conjugated to a protein can affect characteristics critical to its function, including pharmacokinetics, stability, bioactivity, or immunogenicity, it is important in many uses to determine which glycans are present. Thus, the ability to remove some or all of the glycans from a protein and to analyze the glycans or the protein, or both, to determine their composition or compositions is useful for determining whether a protein will have a desired effect. For example, the Food and Drug Administration requires characterization of carbohydrates attached to biologics (such as therapeutic glycoproteins and vaccines) to show composition of matter and consistency of manufacture, resulting in a need for extensive characterization of the product. Analysis of the profile of the released carbohydrates is also important for quality control in the production of recombinant proteins, in which a change in carbohydrate profile may indicate stress in the system, signaling conditions that may require a commercial-scale fermenter of expensive protein to be discarded.

The techniques used to analyze glycans are complex, cumbersome and often time-consuming. The glycans must be released or removed from the protein, a process known as “deglycosylation,” before analysis can be performed. Further, the samples to be analyzed typically include cell lysates, cell culture supernatants, and clinical plasma or serum samples, which may contain a multitude of glycoproteins in addition to the one of interest. Thus, companies wishing to obtain analysis of the carbohydrates attached to a particular glycoprotein, such as an antibody intended for therapeutic use in humans, often have first to perform a number of steps to isolate the target glycoprotein from others in the raw sample.

Only a subset of proteins can be deglycosylated under native, or non-denaturing, conditions, in which the protein is simply mixed with an enzyme that will release from the protein the glycans conjugated to the protein. These methods have the advantage of mild conditions and simple clean up, but often result in incomplete release of glycans. For most proteins being deglycosylated by enzymatic digestion, however, the secondary and tertiary structures of the proteins do not permit access of the enzyme to the carbohydrates unless the protein is first denatured to alter those structures. Traditional protocols for denaturing involve the use of detergents and reducing agents, and an overnight incubation. For example, these protocols typically adding to the glycoprotein a reducing agent such as beta-mercaptoethanol, an anionic detergent, such as sodium dodecyl (lauryl) sulfate, a non-ionic detergent, such as octylphenolpoly(ethyleneglycolether), and a deglycosylating enzyme, and incubating the resulting mixture for 16 hours at 37° C. Once the protein is deglycosylated, the glycans are removed and, usually, are labeled. These protocols are effective and largely independent of the protein (that is, they can be used on most proteins), but are harsh, typically use detergents, which must be removed before some analytical processes can be conducted, and have more clean-up steps.

In commercial cell culture settings, the length of sample processing, typically at least a day, when these standard deglycosylation methods are used reduces the possibility of using carbohydrate analysis as a marker for rapid, in-process analysis and as a tool for control of process variables, such as stress during the cell culture process. The use of detergents adds steps and time since any detergent remaining with the released glycans can interfere with the results of mass spectrometry or other techniques used to analyze the carbohydrate profile

Deglycosylation, in turn, is achieved by one of two methods, chemical release or enzymatic digestion. Most of the available methods for chemical release result in the destruction of the protein backbone of the glycoprotein, and are therefore unsuitable when analysis of the protein component of the glycoprotein is desired. Enzymatic digestion usually occurs under milder conditions and leaves the protein component intact. It is therefore preferable in many analytical situations. Enzymatic digestion is particularly useful for removing N-glycans (glycans linked to the protein through amide groups of asparagine residues), which can be released from glycoproteins by enzymatic cleavage using the exemplar enzyme PNGase F (Peptide-N4-(acetyl-β-glucosaminyl)-asparagine amidase, EC 3.5.1.52) or exoglycosidases or endoglycosidases such as endo-alpha-N-acetyl-galactosaminidase, Endoglycosidase F1, Endoglycosidase F2, Endoglycosidase F3, or Endoglycosidase H. The glycans are then typically treated to label their free-reducing terminus with a fluorescent dye, excess label in removed, and the labeled glycans analyzed by methods such as high performance liquid chromatography (HPLC), capillary electrophoresis (CE), carbohydrate gel electrophoresis. Or the protein component can be analyzed by any of various techniques, including mass spectrometry (MS). Both the enzymatic and the chemical deglycosylation procedures encompass multiple steps, extended incubation times, and clean-up steps prior to analysis of the released glycans.

More recently, various protocols have been developed which reduce the time needed for deglycosylation compared to traditional protocols. For example, U.S. Patent Application Publication US2013/0171658 A1 describes methods of releasing glycans from a target glycoprotein in a biological sample wherein the biological sample is added to a solid support comprising an affinity ligand immobilized in a packed bed, on a monolith, or on a membrane, binding the target glycoprotein, washing away any unbound glycoprotein, and then contacting the bound target with a deglycosylation enzyme to release glycans from the glycoprotein. This protocol permits rapid isolation and deglycosylation of a target glycoprotein even if other glycoproteins in the sample are not known. In 2010, Agilent Technologies announced the introduction of a so-called mAb-Glyco Chip for deglycosylation and analysis of monoclonal antibodies, in which a deglycosylation enzyme is immobilized in a thin capillary through which the monoclonal antibodies of interest are flowed. Agilent's product materials state that the system allows deglycosylation of the monoclonal antibodies in four minutes. These protocols are based, in part, on improving the kinetics of the enzymatic deglycosylation by placing the glycoprotein into close proximity to the immobilized enzyme, as compared to traditional protocols in which both the enzyme and the glycoprotein are in solution and reacting by solution-phase kinetics.

Reagent and device manufacturers have also tried to find reagents which reduce the number of steps or time to complete an analytical workflow. U.S. Pat. No. 7,229,539 discloses degradable surfactants which do not need to be removed following the deglycosylation reaction. According to that patent, once deglycosylation has taken place, the surfactant is degraded by adding an acid to the medium and the resulting products are compatible with MS.

There remains a need for methods of deglycosylation that can be performed rapidly, that reduce handling and clean up steps, and that are suitable for use with multiple analytic methods. Surprisingly, the present invention meets these and other needs.

### BRIEF SUMMARY OF THE INVENTION

The invention provides novel methods of denaturing a glycoprotein of interest in vitro and deglycosylating the glycoprotein by enzymatic digestion. The methods comprise incubating the glycoprotein with a solution comprising an effective amount of a bile detergent, thereby denaturing the glycoprotein, and incubating the glycoprotein with a deglycosylation enzyme for a time sufficient to release the glycans, thereby denaturing and deglycosylating the glycoprotein.

In some embodiments, the bile detergent is a bile acid selected from the group consisting of a glyco- or tauro-form of deoxycholic acid, hyodeoxycholic acid, ursodeoxycholic acid, chenodeoxycholic acid, lithocholic acid, cholanic acid, and cholic acid. In some embodiments, the bile detergent is a bile acid selected from the group consisting of deoxycholic acid, hyodeoxycholic acid, ursodeoxycholic acid, chenodeoxycholic acid, lithocholic acid, cholanic acid, and cholic acid, which bile acid is conjugated to an amino acid other than glycine or taurine. In some embodiments, the amino acid is a D-amino acid. In some embodiments, the bile detergent is a salt of a bile acid selected from the group consisting of deoxycholic acid, hyodeoxycholic acid, ursodeoxycholic acid, chenodeoxycholic acid, lithocholic acid, cholanic acid and cholic acid, or of a glyco- or tauro-form of the bile acid. In some embodiments, the salt of the bile acid bears a cation selected from the group consisting of sodium, lithium, ammonium, and triethylammonium. In some embodiments, the salt is sodium glycodeoxycholate. In some embodiments, the salt is lithium deoxycholate. In some embodiments, the deglycosylation enzyme is selected from the group consisting of an exoglycosidase, an endoglycosidase, and PNGase F. In some embodiments, the deglycosylation enzyme is PNGase F.

In some embodiments, the release of the glycans from the glycoprotein results in forming a solution comprising the glycans, the glycoprotein from which said glycans have been released, and the bile detergent. In some embodiments, the glycoprotein or said deglycosylation enzyme is immobilized on a solid support. In some embodiments, the denatured glycoprotein is immobilized on a solid support prior to being contacted with the deglycosylation enzyme. In some embodiments, the glycans are N-glycans. In some embodiments, the glycans are O-glycans.

In some embodiments, the methods further comprise the steps of heating a solution comprising the glycoprotein and the bile detergent to a temperature ranging from about 80° to about 120° C., maintaining the mixture within said range a period of time T, and then cooling the glycoprotein and the bile detergent. In some embodiments, the glycoprotein and the bile detergent are heated to a temperature from about 90° to about 100° C. In some embodiments, the mixture of the glycoprotein and the bile detergent are cooled to a temperature of about 35° C. to about 60° C. In some embodiments, time T is between about 1 to about 10 minutes. In some embodiments, time T is between about 2 to about 6 minutes. In some embodiments, time T is about 3 minutes. In some embodiments, the released glycans are labeled following release from said glycoprotein. In some embodiments, the released glycans are O-glycosylamines In some embodiments, the label is fluorescent. In some embodiments, the labeled released glycans are analyzed. In some embodiments, the analysis is selected from the group consisting of high-performance liquid chromatography, hydrophilic interaction chromatography, nuclear magnetic resonance, Western blotting, gel electrophoresis, capillary electrophoresis, microfluidic separation, and mass spectrometry. In some embodiments, the method further comprises detecting a fluorescent signal from said labeled released glycans. In some embodiments, the glycoprotein from which glycans have been released is subjected to analysis following the release of said glycans. In some embodiments, the analysis of the glycoprotein from which glycans have been released is selected from the group consisting of high-performance liquid chromatography, hydrophilic interaction chromatography, nuclear magnetic resonance, Western blotting, gel electrophoresis, capillary electrophoresis, microfluidic separation, and mass spectrometry. In some embodiments, the analysis is by mass spectrometry.

In some embodiments, the methods further comprise adding to the solution comprising the released glycans, the glycoprotein from which the glycans have been released and the bile detergent an acid in a quantity sufficient to precipitate the bile detergent, thereby forming a solution containing the glycans and the glycoprotein from which the glycans have been released, and a precipitate containing the bile detergent. In some of these embodiments, the method further comprises separating the solution from the precipitate. In some embodiments, the bile detergent is a bile salt. In some embodiments, the separation is by centrifuging a container holding both said solution and said precipitate to separate said solution from said precipitate, and removing said solution from said container. In some embodiments, the removing of the solution is by pipetting the solution from the container. In some embodiments, the separating is by flowing the solution and the precipitate into a solid phase extraction device which captures the precipitate and allows the solution to flow through. In some embodiments, the acid is an organic acid. In some embodiments, the organic acid is selected from the group consisting of formic acid, acetic acid, a halogenated acetic acid, citric acid, oxalic acid, malic acid, glycolic acid, propionic acid, butyric acid, valeric acid, caproic acid, oxalic acid, lactic acid, benzoic acid, caprylic acid, pelargonic acid, undecylic acid, lauric acid, tridecylic acid, myristic acid, pentadecanoic acid, palmitic acid, margaric acid, stearic acid, nonadecylic acid, and arachidic acid. In some embodiments, the acid is added in a quantity sufficient to reduce the pH of said solution to a pH between 1 and 5.

In some embodiments, the solution further comprises a reductant. In some embodiments, the reductant is tris(2-carboxyethyl)phosphine. In some embodiments, the solution further comprises an alkylant. In some embodiments, the alkylant is iodoacetamide. In some embodiments, the solution comprises an additional detergent. In some embodiments, the solution further comprises an additional organic solvent denaturant. In some embodiments, the additional organic solvent denaturant is selected from the group consisting of acetonitrile, methanol, ethanol, acetone, tetrahydrofuran, trifluoroethanol, and hexafluoroisopropanol. In some embodiments, the bile detergent is removed prior to contacting the denatured glycoprotein with the deglycosylation enzyme. In some embodiments, the bile detergent is removed by adding an acid to the solution in a quantity sufficient to precipitate the bile detergent, thereby forming (a) a solution containing the denatured glycoprotein and (b) a precipitate containing the bile detergent. In some of these embodiments, the method further comprises separating the solution from the precipitate. In some embodiments, the methods further comprise flowing the solution comprising the released glycans, the glycoprotein from which the glycans have been released and the bile detergent into a solid phase extraction device to separate the released glycans from the bile detergent. In some embodiments, the glycoprotein is immobilized on a solid support and the solution comprising the released glycans and the bile detergent is flowed into the solid phase extraction device to separate the released glycans from the bile detergent. In some embodiments, the bile detergent is a bile salt.

### BRIEF DESCRIPTION OF THE DRAWINGS

FIG. 1 is a diagram showing the structure of the bile acid cholic acid, and arrows showing the positions of the three hydroxyl groups present in cholic acid. The labeling of the rings is not shown in the Figure, however, the three cyclohexyl rings are conventionally labeled from left to right as A, B and C, and the cyclopentyl ring is conventionally labeled ring D. In the other bile acids whose names are shown next to the arrows, some or all of the hydroxyl groups present in cholic acid are either absent or have a different position or orientation with respect to the plane of the ring. In ursodeoxy-, chenodeoxy-, and hyodeoxy-bile acids, the hydroxyl group on the C ring is absent. In ursodeoxy-bile acids, the hydroxyl group on the B ring is also inverted with regard to the plane of the ring, while in hyodeoxy-bile acids, the hydroxyl group on the B ring is on the carbon to the left of the one on which it is normally present. In deoxy-bile acids, the hydroxyl group on the B ring is absent. As noted on the Figure, in cholanic acid, all three hydroxyl groups are absent. In glyco- and tauro-bile acids, the amino acid reacts with the carboxylic group on the upper right of the Figure.

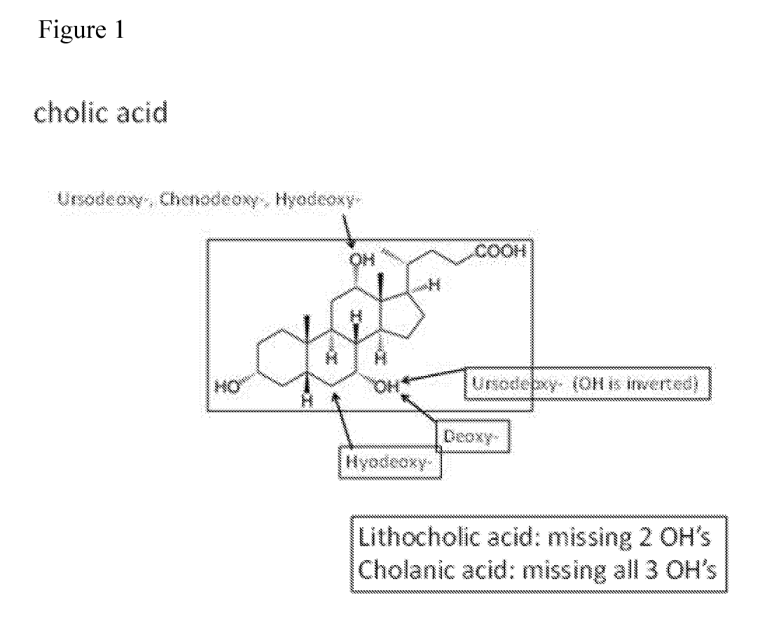

FIG. 2 shows the use of a bile detergent denaturant in an exemplar “rapid labeling” workflow in which glycosylamines released from a glycoprotein by enzymatic digestion are labeled with an anime-reactive dye in the presence of a bile detergent, which detergent is then removed prior to analysis.

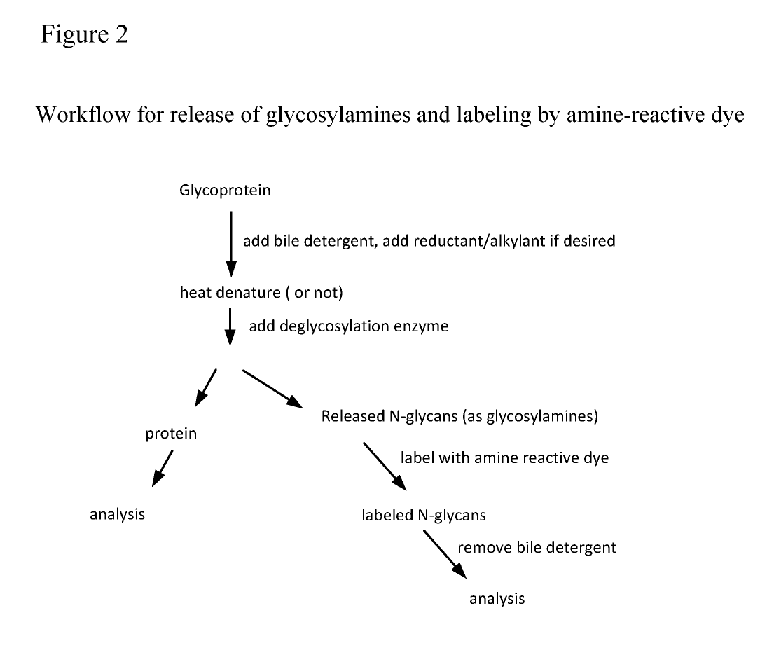

FIG. 3 shows the use of a bile detergent denaturant in an exemplar traditional workflow using reductive amination to label glycosylamines released from a glycoprotein by enzymatic digestion. To avoid interference of the reductive amination by the bile detergent, the bile detergent is removed prior to labeling by precipitating the bile detergent with an acid. Once the bile detergent is removed and the glycans are labeled, they are subjected to analysis.

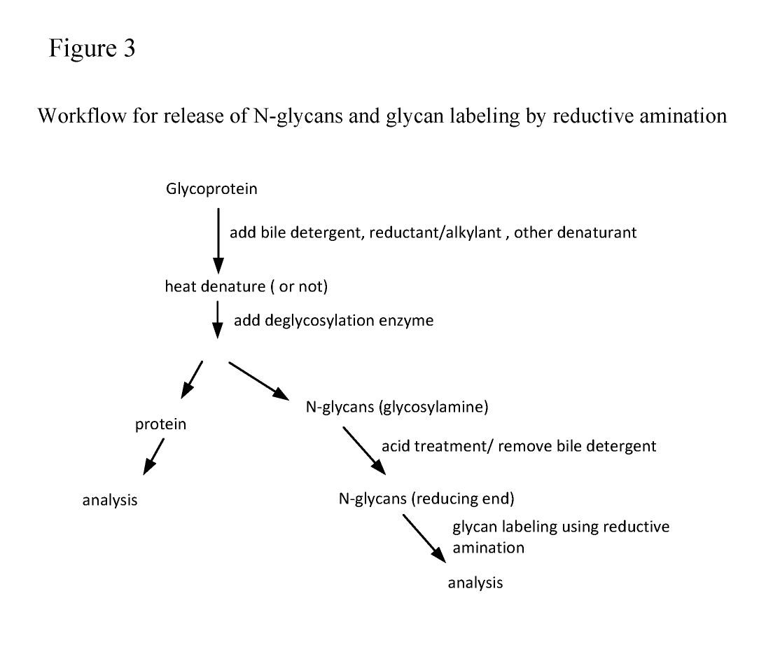

FIG. 4 is a chromatograph of high performance liquid chromatography (HPLC) of labeled N-glycans from human alpha-1-acid glycoprotein from the study reported in Examples 1 and 2. The Y axis shows the signal detected by the instrument, in emission units, or “EU.” The X axis shows the retention time, in minutes.

### DETAILED DESCRIPTION

Analyzing the carbohydrates, or glycans, attached to glycoproteins is important to understanding the pharmacokinetics, immunogenicity, and potential effectiveness of the glycoproteins. Further, analyzing a glycoprotein after some or all of its glycans have been removed has also become important in confirming the glycosylation patterns on a range of proteins, and for confirming the composition of the protein portion of the glycoprotein. For much of the past thirty years, workflows, or protocols, for releasing and analyzing glycans on glycoproteins have used overnight enzymatic digestions to release the glycans from the glycoproteins, followed by labeling and analysis of the released glycans.

Surprisingly, the present invention provides reagents and methods of releasing glycans from glycoproteins that sharply reduce the time needed for workflows in which glycoproteins are denaturated and then deglycosylated by enzymatic digestion, as well as workflows for the analysis of either the released glycans, or of the fully or partially deglycosylated protein component of the glycoprotein, or of both. Advantageously, and surprisingly, the reagents are compatible with use of the exemplar deglycosylation enzyme PNGase F (Peptide-N4-(acetyl-β-glucosaminyl)-asparagine amidase, EC 3.5.1.52), which releases N-glycans from glycoproteins in the form of glycosylamines.

Glycoproteins are typically denatured prior to deglycosylation, particularly when the deglycosylation is being performed using enzymes, to facilitate access of the enzyme to glycosylation sites which may be hidden or blocked by secondary or tertiary structure. The studies underlying the present invention showed that bile detergents, such as bile salts, can be used as agents to denature glycoproteins prior to enzymatic deglycosylation. The glycoprotein used in the studies reported herein was human α-1-glycoprotein, a glycoprotein that is considered resistant to deglycosylation by PNGase F. The studies revealed not only that the bile detergents made the glycans attached to this resistant glycoprotein accessible for deglycosylation by PNGase F, without denaturing the enzyme. And, typical protocols for deglycosylating glycoproteins rely on overnight digestions. The studies reported in the Examples used a digestion time of 5 minutes in conjugation with labeling released glycosylamines with a glycosylamines-reactive dye. Having the denaturant present during the deglycosylation step is advantageous because it helps prevent the glycoprotein from refolding and perhaps rendering some sites once again inaccessible to the enzyme before deglycosylation can occur.

Bile detergents are also particularly useful for denaturation of glycoproteins in analytical workflows because many of them can be precipitated out with a mild acid, which allows them to be quickly and easily removed prior to subjecting the released glycans, or the protein remaining after release of some or all of the glycans, or both, to analysis. This makes bile detergents a more useful denaturant than sodium dodecyl sulfate, which is used as a denaturant in many traditional deglycosylation protocols, but which is difficult to remove and is not compatible with liquid chromatography (LC) or mass spectrometry (MS), which are the most common methods of analyzing what proteins are present. The use of a bile detergent, and particularly of a bile salt, as a denaturant allowed reducing the time to conduct one exemplar current workflow for deglycosylating a glycoprotein with PNGase F and preparing the glycoprotein for analysis from 4 hours to 30 minutes, providing partially or fully deglycosylated glycoproteins which could then be analyzed by LC and MS.

These results could not have been predicted. Bile salts have been used in connection with trypsin hydrolysis of proteins, but trypsin is an enzyme that occurs naturally in the duodenum, a portion of the small intestine into which bile salts are secreted from the pancreas. Trypsin has therefore presumably been selected in the course of evolution for the ability to maintain robust enzymatic activity in the presence of bile salts. PNGase F, on the other hand, is a bacterial enzyme that is not found naturally in the small intestine, has not undergone evolutionary pressure for the capability to be active in the intestinal environment, and which does not appear previously to have been shown to function in the presence of bile salts. Further, trypsin is a serine protease that catalyzes the hydrolysis of peptide bonds, while PNGase F is an amidase hydrolase which cleaves not peptide bonds but rather carbohydrates linked by a nitrogen to an asparagine residue. The two enzymes do not share similar structures, do not share functional properties, and do not catalyze hydrolysis of the same substrate. There was therefore no structural, functional, or activity reason to extrapolate from trypsin's ability to function in the presence of a bile salt that PNGase F would not be denatured or inactivated in the presence of a bile salt.

### Bile Detergents Useful in Denaturing Glycoproteins

The studies underlying the present invention employed salts of the bile acid deoxycholic acid. Like all bile acids, deoxycholic acid has a four ring structure derived from cholesterol; the bile acids vary from one another in whether particular carbon atoms in the rings bear a hydroxyl group and, if so, whether the hydroxyl group is positioned above or below the ring. It is expected that the primary and secondary bile acids, including not only deoxycholic acid but also cholic acid, ursodeoxycholic acid, hyodeoxycholic acid, chenodeoxycholic acid, cholanic acid, and lithocholic acid, are too insoluble to be used as denaturants without modification.

Modifications that make bile acids more soluble, and therefore suitable to be used as denaturants of deglycosylation enzymes, can be accomplished in various ways. In one group of embodiments, one of the primary or secondary bile acids listed in the preceding paragraph can be conjugated with either the amino acid glycine or the amino acid taurine to form a conjugated bile acid. A bile acid that has been conjugated to glycine is designated by the prefix “glyco,” such as “glycodeoxycholic acid.” A bile acid that has been conjugated to taurine is designated by the prefix “tauro,” as in “taurodeoxycholic acid.” It is expected that both the glyco- and the tauro-forms of the conjugated bile acids will be suitable for use as denaturants of deglycosylation enzymes and, in particular, of the exemplar deglycosylation enzyme, PNGase F.

Glyco- and tauro-conjugated bile acids can be readily removed from solution when no longer desired. Glyco-forms of conjugated bile acids can be removed from solution either by any of a number of solid phase extraction columns, by liquid-liquid extraction procedures known in the art, or by acid precipitation, as discussed further below. Tauro-conjugated bile acids can be readily removed from solution by solid phase extraction columns or by liquid-liquid extraction procedures, but they are not expected to be removable from solution by acid precipitation.

In a second group of embodiments, bile acids, including both unconjugated and conjugated bile acids, can be made more soluble by treating the bile acid with a conjugate base to result in a salt. Such salts bear a cation, such as sodium, lithium, ammonium, or triethylammonium. Sigma-Aldrich Corp. (St. Louis, Mo.), for example, sells at least the following sodium salts of bile acids: sodium cholate hydrate, sodium deoxycholate, sodium glycocholate hydrate, sodium taurocholate, and sodium taurodeoxycholate hydrate.

We did a comparison of four different cations to determine whether the result of the denaturation and deglycosylation of a test mixture of glycoproteins differed when a different cation was used to make the bile salt. We found that sodium-, lithium-, ammonium-, and triethylammonium-deoxycholate, each tested at 0.3% v/v, each gave comparable results in the amount of glycans detected and in detection of the glycans known to be present on the test glycoproteins.

A large number of cations are known in the art and are available to make conjugated bile acids or bile salts. It is expected that any non-nucleophilic cation that is 300 Daltons or smaller in molecular weight will be suitable. It is also expected that persons of skill in the art are familiar with the many conjugate bases that can be used to make bile salts. Salts made with imidazole are likely soluble and are expected to provide a cation, but are less preferred because they are expected to interfere with downstream glycosamine labeling using amine-reactive dyes. If imidazole is used to form a bile salt, the bile salt is therefore preferably removed prior to glycan labeling. We found that sodium hyodeoxycholate did not precipitate with acid and should be removed by solid phase or liquid-liquid extraction techniques. By extension, other salts of hyodeoxycholate which have not been conjugated to an amino acid are expected to be removable by solid phase or liquid-liquid extraction techniques, but not by acid precipitation. Any particular salt of any particular primary or secondary bile acid can, of course, be readily tested to determine whether it can be precipitated by acid or needs to be removed by solid phase or liquid-liquid extraction techniques.

In another group of embodiments, the bile acids are modified by being conjugated to a different amino acid than glycine or taurine, the amino acids to which bile acids are conjugated in nature (taurine is not an amino acid in the usual sense, as it has a sulfur group rather than a carboxyl group, but is sometimes treated as one and is treated as one herein). In some embodiments, the amino acid is a naturally occurring amino acid, such as alanine or serine. Amino acids except for glycine are stereoisomers, and most occur in nature only as L-isomers. As the amino acids are not being conjugated to a bile acid for their ability to participate in biological reactions, it is believed that amino acids in the D-form (a “D-amino acid”) can also be used as denaturing reagents in the methods discussed herein. In some embodiments, the amino acid conjugated to the bile acid can be a D-amino acid, and in some embodiments is D-taurine. Some 500 amino acids are known, of which 23 participate in protein-building (are “proteinogenic”) and only 20 of which are encoded by codons. In some embodiments, the D-amino acid conjugated to the bile acid is the D-form of an amino acid which is encoded by a codon. In some embodiments, the D amino acid conjugated to the bile acid is the D-form of an amino acid which is a proteinogenic amino acid.

In general, more soluble conjugated bile acids and bile salts are preferred to less soluble ones. Typically, the conjugated bile acid or bile salt will be used in a concentration of 0.1 to 1.0%, more preferably 0.1 to about 0.9%, still more preferably 0.2 to about 0.8%, and in still more preferred embodiments, about 0.4-0.5%, where the term “about” means plus or minus 0.05% and the concentration is measured as volume/volume. Persons of skill are aware that glycoproteins differ in how hard they are to denature and that this is usually determined empirically. If a particular glycoprotein proves to be hard to denature, the concentration of the buffer is not increased above 0.8%. Typically, glycoproteins that are hard to denature are denatured at a higher temperature, as described in the section on heat of denaturing. In studies underlying the present disclosure, using the preferred bile salts sodium glycodeoxycholate and lithium deoxycholate, good results were obtained at concentrations of 0.4%.

A series of assays were run testing the ability of different bile salts to denature glycoproteins for deglycosylation using the exemplar deglycosylation enzyme PNGase F. The assays used a mixture of glycoproteins, including at least one considered easy to denature and at least one considered hard to denature. The results were reviewed with respect to the “signal” when the glycans released from the glycoprotein were labeled and then analyzed (indicating that the expected amount of glycans present on the glycoproteins was released by the enzymatic digestion) and with respect to “bias” (whether the signal reflected all of the types of glycans known to be present on the sample glycoproteins, in the correct amounts), indicating whether the bile detergents salts were able to denature the hard-to-denature glycoprotein sufficiently to expose al the glycans to the enzyme without inactivating the enzyme. As noted above, good results were obtained with sodium glycodeoxycholate and with lithium deoxycholate at concentrations of 0.4%. Sodium ursodeoxycholate gave a slightly lower signal, but was relatively unbiased. Good results were not obtained with sodium chenodeoxycholate at 0.4%, and that salt is therefore less preferred for use with PNGase F at this concentration. The salt may, however, work well with PNGase F when the salt is used at a higher concentration, or may work well with other deglycosylation enzymes at the same concentration. Studies using different cations showed that denaturing and deglycosylation using sodium-, lithium-, ammonium-, and triethylammonium-deoxycholate at 0.3% v/v resulted in comparable signals and comparable bias.

Testing of particular combinations of reagents for use with particular glycoproteins is common in the art given the diversity of glycoproteins that the practitioner may need to deglycosylate for a particular product or for a particular production run. Determining whether any particular bile salt works well with any particular deglycosylation enzyme on a particular glycoprotein is therefore readily tested by persons of skill.

Glycoproteins are usually denatured in a buffer which already contains one or more salts. For example, phosphate buffered saline is a common buffer which, as its name states, is a saline solution. Some combinations of bile salts in combination with the salt already in the buffer may constitute a high enough concentration of salt as to cause the glycoprotein to precipitate. Where the practitioner intends to perform an assay using a new combination of a particular conjugated bile acid or bile salt and a particular buffer with a particular glycoprotein, it is good practice to combine a small amount of these components to verify that the glycoprotein remains in solution. If the glycoprotein precipitates, which is easily observed visually, that indicates that that particular combination is too salty for use with that glycoprotein and that the practitioner should select a buffer with a lower salt concentration or a different salt. As persons of skill are aware, these kinds of preliminary tests to find combinations of reagents suitable for a particular glycoprotein are usual in this art.

In some embodiments, bile salts are used to denature the glycoprotein of interest. In some embodiments, bile acids are used to denature the glycoprotein. In some embodiments, a mixture of more than one bile salt or of more than bile acid, or a mixture of both a bile salt and a bile acid may be used to denature the glycoprotein. The term “bile detergent” is used herein when reference can include reference to a bile salt, a bile acid, or a combination of a bile salt and a bile acid.

Persons of skill will appreciate that the ease of denaturing glycoproteins depending on a range of factors, including their secondary and tertiary structure, and some glycoproteins are resistant to denaturing even under conditions that would denature most other glycoproteins. In some embodiments, particularly with regard to denaturing a glycoprotein known to be hard to denature or one proving in practice to be hard to denature completely using a bile detergent, the practitioner may wish to also add one or more additional detergents or additional organic solvent denaturants, such as acetonitrile, methanol, acetone, ethanol, tetrahydrofuran or guanidinium chloride. These additional detergents or denaturants can conveniently be removed by use of a suitable cleanup column. Persons of skill are knowledgeable about the use of these reagents and their removal after they have served their purpose.

### Other Agents That May be Used During Denaturation of the Glycoprotein

The solution containing the glycoprotein and the bile detergent can contain further agents commonly used in protocols for deglycosylating glycoproteins. In particular, the solution can contain reductants, such as tris(2-carboxyethyl)phosphine, dithiothreitol (DTT), beta-mercaptoethanol (BME), alkylants, such as iodoacetamide, or a combination of reductants and alkylants. It is expected that persons of skill in denaturing and deglycosylating glycoproteins are familiar with the use of each of these types of reagents and the compounds usually used for these purposes.

### Heating the Glycoprotein-Bile Detergent Mixture to Speed Denaturation

To denature the glycoprotein, a solution containing the bile detergent of choice is added to the glycoprotein and the resulting mixture is incubated. In some embodiments in which the practitioner want to complete the denaturation more quickly, the mixture can be heated and in some embodiments, the mixture is heated. In some embodiments, the mixture is heated to a high temperature (typically, 90° C., although for glycoproteins known to be hard to denature, it may be higher). In some embodiments, the mixture is heated as high as 100° C. In most embodiments, the mixture will not be heated higher than 100° C., although in some embodiments, the mixture may be heated as high as 120° C. Typically, the solution will be heated for a time between 1 minute and about 10 minutes, more preferably 2-7 minutes, still more preferably about 2-5 minutes, even more preferably about 3 to about 5 minutes and most preferably about 3 minutes. It is not expected that heating the mixture for more than about 5 minutes will improve the denaturation of the glycoprotein. As used herein in connection with a statement of a time, the term “about” means plus or minus 30 seconds.

### Cooling

The denatured glycoprotein will preferably be cooled before being deglycosylated, both to avoid denaturing the enzyme and to permit the deglycosylation to occur at a temperature in a range at which the enzyme is most active. The solution containing the glycoprotein is preferably cooled to about 22-60° C., and more preferably about 35-55° C. In some preferred embodiments, the glycoprotein is cooled to about 50° C. Persons of skill will appreciate that for some equipment, the heat transfer from the apparatus to the reactants is not complete and that a temperature setting of the heating apparatus will result in the reactants being at a temperature several degrees cooler than the temperature setting, and will adjust accordingly. In studies reported herein, the deglycosylation was conducted with the apparatus set to 50 ° C. As used herein in connection with a temperature, the term “about” means plus or minus 1 degree C.

### Deglycosylation

In some embodiments, the glycoprotein is deglycosylated by a deglycosylation enzyme. In some embodiments, the deglycosylation enzyme is an exoglycosidase or an endoglycosidase such as Endoglycosidase F1, Endoglycosidase F2, Endoglycosidase F3, or Endoglycosidase H. In some embodiments, the deglycosylation enzyme is PNGase F. In some embodiments, the glycans to be deglycosylated enzymatically are O-glycans and the enzyme used for deglycosylation is O-glycosidase, alone or in combination with an appropriate mix of exoglycosidases. In some embodiments, the practitioner wishes to distinguish between any N-glycans that may be present on the glycoprotein from any O-glycans that may be present. In some embodiments, the enzymes mentioned above are used in connection with a bile detergent denaturation to provide a fast method of removing the N-glycans from the glycoprotein so that any O-glycans or glycosaminoglycans (GAGs) that may be on the glycoprotein can be analyzed. For example, the first digestion may be made using an enzyme to remove N-glycans, followed by a second enzymatic digestion with endo-alpha-N-acetyl-galactosaminidase to remove O-glycans. It is expected that persons of skill are familiar with the various enzymes used for enzymatic release of glycans and the glycans released by each. Any particular enzyme can be readily tested to determine its compatibility for use with any particular bile detergent by substituting it for PNGase F in the exemplar protocol set forth in the Examples.

### Labeling

The exemplar deglycosylation enzyme is PNGase F, which releases the glycans as glycosylamines If the glycosylamines are to be labeled with an amine-reactive dye, the dye labeling can be conducted without removal of the bile detergent. If labeling is to be performed using reductive amination of the reducing end of the glycan, the bile detergent is preferably first removed. Regardless of the method of labeling, the bile detergent will also be removed before subjecting the labeled or unlabeled glycoprotein to those analytical methods, such as mass spectrometry, in which the presence of the bile detergent would be incompatible or would be a confounding factor.

### Removal of the Bile Detergent

The bile detergent can be removed by any convenient means known in the art. In one convenient group of embodiments, the bile detergent is removed by precipitating the bile detergent with an acid, leaving the glycoprotein and the glycans in the supernatant. If desired, the amount of acid needed to precipitate a bile detergent of choice can be determined before the assay by, for example, adding measured amounts of the acid to a container of a known amount of the bile detergent until precipitation is observed.

As noted, upon precipitation of the bile detergent, the denatured glycoprotein and any glycans remain behind in the supernatant. In some embodiments, the precipitate is removed, leaving behind the supernatant. In most cases, however, it will be more convenient to remove the supernatant from the container, leaving the precipitated detergent behind. The supernatant can be removed by any of a number of means known in the art, such as pipetting the supernatant, opening the entrance to a microfluidic channel and flowing the supernatant into the channel and to an analytic means, or by applying suction to vacuum up the supernatant while leaving the precipitate behind.

In a second group of embodiments, the bile detergent is removed by using solid- or liquid-phase techniques. It is expected that persons of skill are familiar with various types of liquid-liquid techniques and solid phase extraction devices which are used in the art to remove detergents from a solution. The solid phase extraction devices usually comprise resins on a solid support, and the resins are conveniently disposed in a cartridge or column (for convenience of reference, reference to a “column” in the following discussion refers to either a column or a cartridge, unless otherwise required by context). Common solid phase extraction devices include: reverse phase columns, normal phase cartridges or columns, ion exchange columns, and size exclusion columns Typically, the cleanup columns bind the glycans, allowing the detergent to flow through and be discarded, after which the glycans are eluted from the column. The use of a solid- or liquid-phase extraction technique is preferred when the bile detergent is a tauro bile acid or tauro bile salt, or a hyodeoxycholate, as these are not susceptible to acid precipitation, and for any other bile detergents that cannot be precipitated by adding acid to the solution.

In a third group of embodiments, the bile detergent is precipitated with an acid and the supernatant is run though a cleanup column to allow the glycans to bind to the stationery phase. Any excess or unbound dye runs through the column and can be discarded. The glycans are then eluted from the column with water and put in an analytical column or subjected to MS. Alternatively, the bile detergent can be precipitated, filtered by a filter column to allow the solution to pass through while the precipitate is captured in the filter column, and the filtrate passed into a cleanup cartridge to allow removal of excess free dye, protein, enzyme, salt, buffer, or other undesired reagents before providing the glycans to an analytical column or MS. If one or more denaturants are used in addition to a bile detergent, they are typically removed by using a cleanup column, such as a solid phase extraction column, and do not have to be LC or MS compatible.

Strong inorganic acids, such as hydrochloric acid, at low concentrations can be used for precipitating the bile detergent. When using acids, particularly strong inorganic acids such as hydrochloric acid, enough should be used to lower the pH below 5, but not below 1, as pH levels below pH 1 are expected to hydrolyze the glycans. In preferred embodiments, the acid is a mild acid and in more preferred embodiments is an organic acid. Suitable organic acids include formic acid, acetic acid, halogenated acetic acids, such as di, tri-fluoro, or chloro acetic acid, citric acid, oxalic acid, malic acid, glycolic acid, propionic acid, butyric acid, valeric acid, caproic acid, oxalic acid, lactic acid, benzoic acid, caprylic acid, pelargonic acid, undecylic acid, lauric acid, tridecylic acid, myristic acid, pentadecanoic acid, palmitic acid, margaric acid, stearic acid, nonadecylic acid, and arachidic acid.

### EXAMPLES

### Example 1

This Example sets forth abbreviations for some of the reagents used in an exemplar workflow of a deglycosylation procedure performed using a bile salt as a denaturant.

Abbreviations for reagents used in the workflow:
HEPES: 4-(2-hydroxyethyl)-1-piperazineethanesulfonic acid
TCEP: tris(2-carboxyethyl)phosphine
TMD: 1,3-Di(4-pyridyl)propane
DMSO: dimethyl sulfoxide
DMF: dimethylformamide

### Example 2

This Example sets forth an exemplar workflow of a deglycosylation procedure performed using a bile salt as a denaturant.

### Denaturation Step:

A 1 μl aliquot of 750 mM HEPES buffer at pH 8 was added to 10-20 μl of a solution of 2 mg/ml desalted human α-1-acid glycoprotein (20-40 μg of protein). TCEP was added at 10 mM (1 μl) and 2 μl of 5% lithium deoxycholate was added to the mixture. The reagents were thoroughly mixed and then heated to 90° C. for 3 minutes to denature the glycoproteins. The sample was cooled to room temperature for 2 minutes.

### Digestion Step:

A 1 μl aliquot of 1 mg/ml PNGase F was added to each of the cooled samples and the samples were incubated at 50° C. for 5 minutes to release the N-glycans. The samples were cooled to room temperature for 2 minutes.

### Labeling Step:

A 1:1 mixture of 0.2 M 2-diethylaminoethyl 4-[(2,5-dioxopyrrolidin-1-yl)oxycarbonylamino]benzoate in DMSO:1 M TMD in DMF was freshly prepared. Five μl of the mixture was added to the sample to label the released glycosylamines The labeling was allowed to proceed for 2 minutes.

### Cleanup Step:

The labeled glycans were resuspended in 200 μl of acetonitrile contining 1% formic acid and the solution was loaded onto GlykoPrep® Cleanup Cartridges (ProZyme, Inc., Hayward, Calif.). The cartridges were spun at 300×g for 3 minutes. The cartridges were washed with 200 μl of a 1:3:96 formic acid:water:acetonitrile solution for an additional 3 minutes at 300×g. The samples were eluted with 50 μl of a solution of 200 mM ammonium formate, pH 7, containing 20% DMSO. One μl of eluted sample was injected for high performance liquid chromatography (HPLC) analysis.

### Example 3

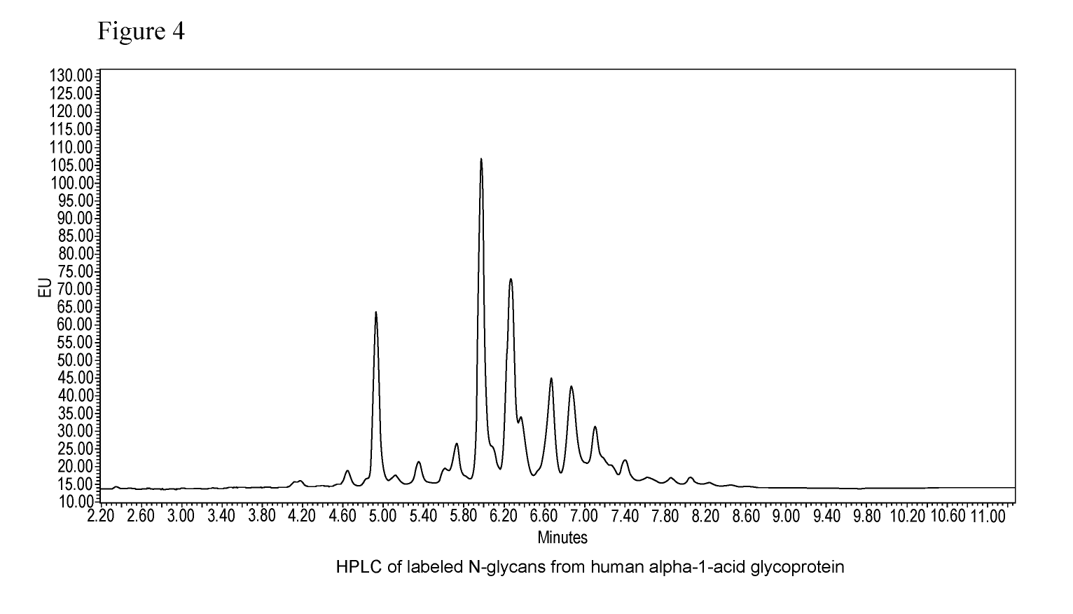

FIG. 4 shows a HPLC chromatogram analyzing the N-glycans obtained from the studies reported in Examples 1 and 2, with emissions units (EU) shown on the Y axis and retention time, in minutes, shown on the X axis.

It is understood that the examples and embodiments described herein are for illustrative purposes only and that various modifications or changes in light thereof will be suggested to persons skilled in the art and are to be included within the spirit and purview of this application and scope of the appended claims. An publications, patents, and patent applications cited herein are hereby incorporated by reference in their entirety for an purposes.

1. A method of denaturing and deglycosylating a glycoprotein of interest in vitro, said method comprising:
(i) incubating said glycoprotein with a solution comprising an effective amount of a bile detergent, thereby forming a denatured glycoprotein, and
(ii) incubating said denatured glycoprotein with a deglycosylation enzyme for a time sufficient to release glycans from said denatured glycoprotein, thereby denaturing and deglycosylating said glycoprotein of interest.

2. The method of claim 1, wherein said bile detergent is a bile acid selected from the group consisting of a glyco- or tauro-form of deoxycholic acid, hyodeoxycholic acid, ursodeoxycholic acid, chenodeoxycholic acid, lithocholic acid, cholanic acid, and cholic acid.

3. The method of claim 1, wherein said bile detergent is a bile acid selected from the group consisting of deoxycholic acid, hyodeoxycholic acid, ursodeoxycholic acid, chenodeoxycholic acid, lithocholic acid, cholanic acid, and cholic acid, which bile acid is conjugated to an amino acid other than glycine or taurine.

4. The method of claim 1, wherein said bile detergent is a salt of a bile acid selected from the group consisting of deoxycholic acid, hyodeoxycholic acid, ursodeoxycholic acid, chenodeoxycholic acid, lithocholic acid, cholanic acid and cholic acid, or of a glyco- or tauro-form of said bile acid.

5. The method of claim 4, wherein said salt of the bile acid bears a cation selected from the group consisting of sodium, lithium, ammonium, and triethylammonium.

6. The method of claim 5, wherein said salt is sodium glycodeoxycholate.

7. The method of claim 1, further wherein said deglycosylation enzyme is selected from the group consisting of an exoglycosidase, an endoglycosidase, and PNGase F.

8. The method of claim 7, further wherein said deglycosylation enzyme is PNGase F.

9. The method of claim 1, further comprising the steps of heating a solution comprising said glycoprotein and said bile detergent to a temperature range of from about 80° to about 120° C., maintaining said mixture within said range a period of time T, and then cooling said glycoprotein and said bile detergent.

10. The method of claim 9, wherein said glycoprotein and said bile detergent are heated to a temperature from about 90° to about 100° C.

11. The method of claim 9, further wherein said cooling of said solution comprising said glycoprotein and said bile detergent is to a temperature of about 35° C. to about 60° C.

12. The method of claim 9, further wherein said time T is between about 1 to about 10 minutes.

13. The method of claim 9, further wherein said glycans are labeled following release from said glycoprotein.

14. The method of claim 13, further wherein said label is fluorescent.

15. The method of claim 13, further wherein said labeled released glycans are analyzed, wherein said analysis is selected from the group consisting of high-performance liquid chromatography, hydrophilic interaction chromatography, nuclear magnetic resonance, Western blotting, gel electrophoresis, capillary electrophoresis, detection of a fluorescent signal from said labeled glycans, and mass spectrometry.

16. The method of claim 1, further wherein said glycoprotein from which glycans have been released is subjected to analysis selected from the group consisting of high-performance liquid chromatography, hydrophilic interaction chromatography, nuclear magnetic resonance, Western blotting, gel electrophoresis, capillary electrophoresis, and mass spectrometry.

17. The method of claim 1, further wherein said release of said glycans forms a solution comprising said released glycans and said bile detergent.

18. The method of claim 17, further comprising adding to said solution comprising said released glycans and said bile detergent an acid in a quantity sufficient to precipitate said bile detergent, thereby forming a solution containing said glycans and a precipitate containing said bile detergent.

19. The method of claim 18, further comprising separating said solution from said precipitate.

20. The method of claim 19, wherein said separation is by centrifuging a container holding both said solution and said precipitate to separate said solution from said precipitate, and removing said solution from said container.

21. The method of claim 19, wherein said separating is by flowing said solution and said precipitate into a solid phase extraction device which captures said precipitate and allows said solution to flow through.

22. The method of claim 18, wherein said acid is an organic acid.

23. The method of claim 18, wherein said acid is added in a quantity sufficient to reduce the pH of said solution to a pH between 1 and 5.

24. The method of claim 1, further wherein said solution comprises a reductant.

25. The method of claim 1, wherein said solution further comprises an alkylant.

26. The method of claim 1, wherein said solution further comprises an additional organic solvent denaturant.

27. The method of claim 1, further comprising removing said bile detergent from said solution and then releasing glycans from said denatured glycoprotein by incubating said denatured glycoprotein with a deglycosylation enzyme for a time sufficient to release said glycans, thereby forming a solution comprising said glycans.

28. The method of claim 27, wherein said bile detergent is removed by adding an acid to the solution in a quantity sufficient to form precipitated bile detergent, and separating said solution from said precipitated bile detergent.
